(19) United States
(12) Patent Application Publication
(10) Pub. No.: US 2005/0106608 A1
(43) Pub. Date: May 19, 2005

(54) Method and apparatus for DNA collection

(76) Inventors: Jangbir Sangha, Overland Park, KS (US); James William Schumm, Vienna, VA (US); John Carter Fox, Washington, DC (US); Robert Arley Bever, Chantilly, VA (US)

Correspondence Address: SHUGHART THOMSON & KILROY, PC, 120 WEST 12TH STREET, KANSAS CITY, MO, 64105, US

(21) Appl. No.: 10/969,034

(22) Filed: Oct. 20, 2004

Related U.S. Application Data
(62) Division of application No. 10/020,257, filed on Dec. 14, 2001.

(51) Int. Cl. C12Q 1/68; C12M 1/34

(52) U.S. Cl. 435/6; 435/287.2

(57) ABSTRACT

A device for collection of DNA-containing materials is provided and a method of preventing DNA specimen contamination is provided which prevents extraneous DNA found in DNA evidence collection kits from interfering with DNA specimen analysis, further a device an method is provided for collecting the DNA-containing material of a subject from the subject's skin.

### FIELD OF THE INVENTION

The present invention relates to evidence collection kits. Specifically, the present invention relates to evidence collection kits used for obtaining sexual assault evidence, DNA evidence, or fingerprint evidence. In the case of DNA evidence, the present invention serves to ensure that extraneous or non-specimen DNA is disabled from being replicated intact during the analysis of specimen DNA.

### BACKGROUND OF THE INVENTION

There are many occasions when it is desirable to collect DNA evidence or DNA information from a crime suspect or victim or other subject at crime scene or from the general population. One of the common situations of DNA collection is after a sexual assault using a sexual assault evidence collection kit. The object of the sexual assault evidence collection is to isolate fibers, pubic hairs, body fluids and any other evidence which may be contained on the body or the clothes of the sexual assault victim. In particular, in sexual assault cases, it is of critical importance to properly collect bodily fluids left by the assailant. The body fluids of the assailant can be used to extract and analyze the DNA of the assailant. The analyzed DNA can potentially provide positive identification of the individual who is suspected of committing the assault.

Another occasion in which it may be useful to collect a DNA sample is upon the detention of a suspect in a crime. DNA samples and DNA sample banks are becoming increasingly important in identifying perpetrators and in clearing potential suspects in cases in which the DNA of the perpetrator was left at the crime scene. In the arrest situation, it would be useful for the officer to be able to collect a DNA specimen from the suspect without the need of the officer to enter into a confrontational situation with the suspect. It also would be beneficial to avoid a situation in which the officer is placed in danger of injury or contamination by the bodily fluids of a suspect. For example, a typical means of DNA collection is the collection of cells and/or saliva from the mouth. Attempting to collect such a sample from an uncooperative subject can place the officer in the situation of attempting to force the mouth open of the subject, to place the subject in a position in which he could head-butt the officer or kick at the officer or place the officer in the situation of being spit upon by the subject thereby making the officer subject to whatever communicable diseases the suspect may carry.

Yet another problem that occurs with current DNA sample collection and DNA testing kits and sexual assault evidence collection kits is the existence and potential for existence of extraneous DNA on the kit collection devices and/or kit packaging and/or the packaging of the collection devices contained within the kit. Such extraneous DNA can enter the kit during the assembly of the kit components. DNA collection kits are assembled by workers handling and packing the components who may or may not be wearing gloves or face masks or hairnets. The use of gloves and face masks and hairnets could reduce the entry of extraneous DNA from the kit packers. In most situation, the workers are not wearing gloves as the individual collection swabs are generally presented to the kit packers in a sealed wrapper that previously has been sterilized in some manner. The kit worker then picks up the materials to go into the kit, puts them in the housing box and closes the sexual assault evidence collection kit. As a typical sexual assault evidence kit contains fifteen to eighteen different items and the envelopes within which those collection items are housed, a multiplicity of kit components are presented which can contact the kit packing workers hands or arms and become contaminated with DNA from the kit packer by coughing or sneezing or from hair falling into the kit. Another source of contamination is presented by the supplier of components to the company packaging the kit. The workers that prepare the component parts of the kit also can present a source of contamination. Therefore, it would be beneficial to DNA evidence collection if a means existed for eliminating such extraneous DNA or non-specimen DNA from presenting a possible source of contamination to the analysis of the actual specimen DNA collected with a sexual assault evidence collection kit or other type of DNA collection kit.

Yet another problem presented by current DNA collection techniques is the difficulties presented by the need to collect DNA samples from, usually, the cheek walls on the inside of the mouth. While this collection technique is quite suitable for cooperative subjects, it presents a number of problems for the collection of DNA samples from uncooperative subjects such as criminal suspects or persons under the influence of alcohol or drugs. As previously indicated, the collection of saliva or cells from the cheek walls inside the mouth requires that the subject voluntarily open their mouth or that personnel be available to force open the mouth of the subject. In that situation the subject is in a position to spit or cough on the person attempting to collect the sample, also the subject is in a position to kick or bite the individual attempting to collect the DNA sample. Therefore, in view of these problems of the prior art DNA sample collection devices including sexual assault evidence collection kits and other DNA collection kits, it would be beneficial if a means of collecting DNA existed which could assure the elimination of extraneous DNA on the collection kit materials once the kit materials have been packed into their container.

Yet another benefit for a DNA evidence collection kit would be that the means of DNA collection avoid confrontational situations between the individual collecting DNA and the subject from whom DNA is being collected.

Another benefit which would be useful in a DNA collection device or evidence collection kit is that the instructions for use of the device be easily understood by the individuals making the sample collection.

Still another benefit that should be offered by a DNA collection device is that the device be rugged and provide fool-proof collection of DNA as well as assurance of adequate sample collection for DNA analysis.

Another benefit in a DNA collection device would be that the device offers rapid collection of a DNA sample from a suspect and that a minimum of paper work is presented to the DNA sample collector.

From the standpoint of a subject from whom a DNA sample is being collected, it would be beneficial if the device for collection was non-invasive and painless. Another benefit for the subject during DNA collection would be that the method of DNA collection is non-confrontational and non-insulting to the subject. Another benefit to a subject from whom DNA is being collected is that the device for means used to collect the DNA sample is safe to the subject.

### SUMMARY OF THE INVENTION

The foregoing and other objects are intended to be illustrative of the invention and are not meant in a limiting sense. The objects of the present invention can be provided for in a collection kit and a method of preparation of a DNA collection kit in which the kit components are assembled into a closure or housing after which the housing containing the kit components is exposed to an effective quantity of an agent for disabling DNA from interfering with subsequent specimen specific DNA analysis.

The present invention also provides a method of collection DNA-containing substances from the skin surfaces of a subject and in one embodiment allows the collector to avoid the confrontational situation of collection from the subject's mouth.

Further objects of the present invention are achieved by an embodiment which permits the collection of both a DNA specimen and a combination fingerprint specimen and DNA specimen from the subject and which allows use of the fingerprint specimen for DNA analysis of the subject's DNA.

Many possible embodiments of the invention may be made and will be readily evident upon a study of the following specification and accompanying drawings comprising a part thereof. Various features and subcombinations of the invention may be employed without reference to other features and subcombinations. Other objects and advantages of this invention will become apparent from the following description taken in connection with the accompanying drawings, wherein is set forth by way of illustration and example, an embodiment of this invention.

### DESCRIPTION OF THE DRAWINGS

Preferred embodiments of the invention, illustrative of the best modes in which the applicant has contemplated applying the principles, are set forth in the following description and are shown in the drawings and are particularly and distinctly pointed out and set forth in the appended claims.

FIG. 1 is a perspective view of one embodiment of the present invention having a collection substrate and a resilient member with openings therein,

FIG. 2 is a perspective view of another embodiment of the present invention having a collection substrate and a resilient member with openings therein and a protective layer above the collection substrate,

FIG. 3 is a perspective view of another embodiment of the present invention having a collection substrate and a protective layer above the collection substrate and a data portion attached to the collection portion,

FIG. 4 is an exploded side elevation view of the embodiment of FIG. 3,

FIG. 5 is a perspective view of another embodiment of the present invention having a collection substrate square and a protective layer above the collection substrate and a data portion attached to the collection portion

FIG. 6 is an alternate view of the device of FIG. 5 and having the collection portion bent to assist in sample collection,

FIG. 7 is a perspective view of another embodiment of the present invention having a multiple collection substrate areas and a protective layer above the collection substrate and a data portion attached to the collection portion,

FIG. 8 is a perspective view of another embodiment of the present invention having an absorbent collection substrate and a protective layer above and below the collection substrate and a data portion attached to the collection portion,

FIG. 9 is an exploded side elevation view of the embodiment of FIG. 8,

FIG. 10 is a perspective view of another embodiment of the present invention having an absorbent collection substrate with a protective layer above and a swab for transfer of a liquid sample to the substrate,

FIG. 11 is a plan view of another embodiment of the present invention having an absorbent collection substrate underneath a cover sheet and a protective backing behind the collection substrate,

FIG. 12a is a side view of the embodiment of FIG. 11 after the device is folded in half for insertion into a subject's mouth,

FIG. 12b is a cross-sectional view of the embodiment of FIG. 11 showing the absorbent collection substrate between the cover sheet and the protective backing,

FIG. 13 is a perspective view of an embodiment of the present invention having an absorbent collection substrate attached to a backing and a protective layer below the collection substrate and a data portion attached to the collection portion,

FIG. 14a is a perspective view of an embodiment of the present invention in the form of a retractable collection substrate housed in a case and with the substrate retracted,

FIG. 14b is a view of an embodiment of FIG. 14a with the substrate extended,

FIG. 15 is a perspective view of an embodiment of the present invention having an adhesive or sticky collection substrate on a backing and with a protective layer above the collection substrate, and

FIG. 16 is an elevation view of the embodiment of FIG. 15;

FIG. 17 is a plan view of another embodiment of the present invention having a fingerprint portion and a specimen portion that can be used to collect a skin swipe sample or an oral sample of the subject's DNA-containing material; and

FIG. 18 is an elevation view of the embodiment of FIG. 17.

### DESCRIPTION OF THE PREFERRED EMBODIMENT

Referring now to FIG. 1, an embodiment of the present invention is shown which relies upon a slightly tacky or adhesive substrate in connection with a more resilient abrasive surface to collect and capture DNA and DNA-containing material from the skin surfaces of a subject. Still referring to FIG. 1, collection device 10 is comprised of handle base 12 which is adapted to receive resilient member 14 therein. Resilient member 14 is held in place in handle base 12 by post 16 which captures securing void 18 in resilient member 14. Positioned on top of resilient member 14 is collection substrate 20. Collection substrate 20, in the embodiment of FIG. 1, is equipped with adhesive face 22 which is positioned adjacent resilient member 14. It will be appreciated by those skilled in the art that substrate 20 also could be either an absorbent material having a non-sticky surface as well. Adhesive face 22 of collection substrate 20 can be any of a number of different substances which present a sticky or adhesive surface and which will attract and retain skin surface cells and DNA material that have been loosened by resilient member 14 for collection on adhesive face 22. By way of example and not limitation, absorbent collection papers such as Whatman FTA, S&S IsoCode®, S&S 903®, or S&S 900® are suitable for use as collection substrate 20, either as manufactured or after treatment to have a sticky or adhesive surface. In general, any clean, absorbent sufficiently strong paper to which a sticky or adhesive surface is applied would be suitable. Resilient member 14 and collection substrate 20 are held in place within handle base 12 by handle cap 22. Handle cap 22 presses into handle base 12 to retain resilient member 14 and collection substrate 20 in place on post 16 by capture of securing void 18 of resilient member 14 and securing void 24 of collection substrate 20.

In operation, the embodiment of FIG. 1 is used to collect skin surface cells from a subject. The embodiment of FIG. 1 is employed by a user by grasping handle base 12 of device 10 which will contain, in the assembled version, handle cap 22. The user, if a person other than the subject, then applies pressure with a gloved finger to the top of collection substrate 20 and rubs the bottom surface of resilient member 14 on the skin of the subject from whom a sample is to be collected. As resilient member 14 is rubbed across the skin surface of the subject, openings 26 serve to abrade skin cells from the surface of the subject's skin and the skin cells that are loosened contact and stick to adhesive face 22 of collection substrate 20 as openings 26 expose several portions of adhesive face 22 to contact with the skin surface and allow the exposed portions of adhesive face 22 to capture dislodged skin cells within the adhesive on adhesive face 22. After skin cells containing DNA have been collected in this manner, the embodiment of FIG. 1 is then placed into a suitable transportation container such as an envelope or a plastic container or plastic sleeve which can be placed over the end of handle base 12 to cover both resilient member 14 and collection substrate 20. For evidentiary purposes, it is then desirable and appropriate to seal the container with a tamper-proof seal such as an adhesive tape or other commonly employed tamper evidence seal prior to shipping the container containing device 10 to a laboratory for specimen analysis.

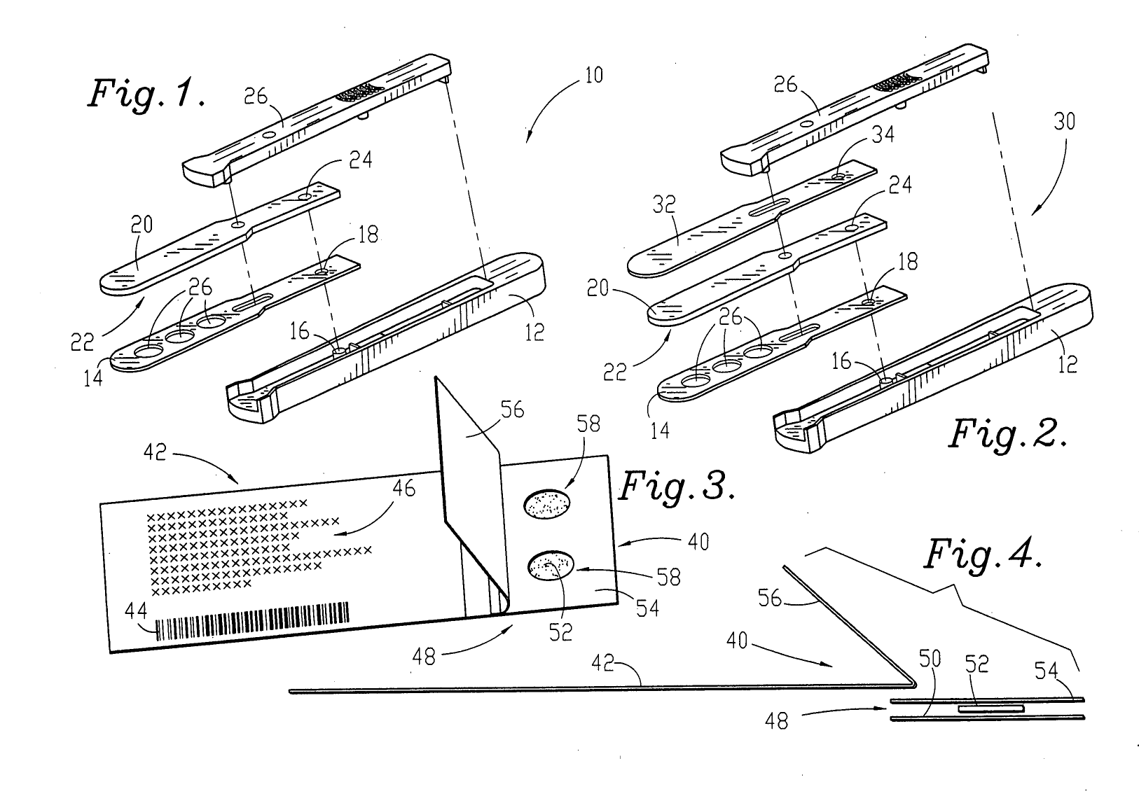

Referring now to FIG. 2, an alternative embodiment of the present invention is shown. In the embodiment of FIG. 2, a structure similar to that of the embodiment shown in FIG. 1 is shown. In the embodiment of FIG. 2, protective layer 32 is positioned directly above collection substrate 20. Protective layer 32 is held in place within handle base 12 by means of post 16 capturing protective layer 32 by securing void 34 in protective layer 32. Protective layer 32 operates to prevent contamination of collection substrate 20 with the extraneous DNA of the sample collector if the sample collector is not the subject or forgets to wear gloves. During the course of collecting DNA, it may be useful for the collector to grasp collection device 30 by handle base 12 and to use the collector's index finger to press against collection substrate 20 to guide the resilient member over the skin surface of the subject from whom DNA is to be collected. If the index finger of the operator or user of collection device 30 were to contact collection substrate 20, extraneous DNA would be introduced into the analysis process. Therefore, by applying protective layer 32 to the upper surface of collection substrate 20, which is not used for collecting DNA, the user of device 30 can press against collection substrate 20 and guide resilient member 14 over the surface of the skin.

The embodiment of FIG. 2 also operates to allow collection of DNA specimens from a particular location while avoiding collection of DNA from adjacent surfaces. In the use of the embodiment of FIG. 2, it will be appreciated that protective layer 32 which is adjacent to the upper surface of collection substrate 20 not only protects collection substrate 20 from receiving DNA-containing material off the fingers of the use, but protective layer 32 will prevent the collection of DNA-containing material from adjacent tissue surfaces during the collection procedure. For example, if material containing DNA was being collected from the mouth of a subject and it was desired to collect from the cheek area while avoiding the teeth and gums, the embodiment of FIG. 2 would allow this selective collection to be accomplished as the collecting surface of substrate 20 is on a first side of substrate 20 and protective layer 32 is against a second side of substrate 20 and preventing the collection of DNA-containing material-thereon.

Referring now to FIGS. 3 and 4, an alternative embodiment of the present invention is shown. Referring now to FIG. 3, DNA collection device 40 is a paper sheet form having a data portion 42 comprising a bar code 44 and area for additional user written information 46. Connected to data portion 42 is DNA collection portion 48. Referring now to FIG. 4, the construction of DNA collection portion 48 will be described. DNA collection portion 48 is a sandwich of materials which is attached to data portion 42. The sandwich of materials comprising DNA collection portion 48 comprises backing paper 50 upon which is placed the selected collecting substrate 52 which is then enclosed by cover sheet 54. This sandwich of materials is attached to data portion 42. A protective layer 56 also is attached to data portion 42 and positioned so that it can overlay DNA collection portion 48 to act as a protective covering for collecting substrate 52.

Referring now to FIG. 3, it is shown that on DNA collection portion 48, voids 58 are provided in cover sheet 54 to expose a portion of collecting substrate 52. In operation, a user wishing to collect a DNA sample from a subject will fold back protective layer 56 to expose cover sheet 54 and collecting substrate 52. Depending on the type of collecting substrate included in device 40, different methods of DNA collection could be employed. For example, if collecting substrate 52 is a sticky or adhesive substance, the user or operator could simply press the area of DNA collection portion containing voids 58 against the surface of the skin of the subject to collect DNA containing material. If, on the other hand, device 40 contained a collecting substrate such as an absorbent paper, the absorbent paper could be rubbed against the subject to collect the DNA sample. If desired, to increase the collection of DNA-containing material, the absorbent material of the collecting substrate or the collection area on the subject may be pre-wetted with a solution which does not disable specimen DNA or interfere with the DNA analysis. Examples of suitable wetting agents are cell-lysing detergents, water or saline or aqueous or non-aqueous solutions of alcohol or an antiseptic solution. The wetting agent can be either lipophilic or non-lipophilic.

The collection substrate for the embodiments of the present invention can present several types of surfaces all of which will be suitable for DNA specimen collection and some of which will be suitable for both DNA specimen collection and fingerprint collection. By way of example and not limitation, cellulose or paper types of substrates such as IsoCode®) or S&S903® or S&S 300® manufactured by Schleicher and Schull, or FTA® manufactured by Whatman, or their equivalents which also are manufactured by Whatman are illustrative of the useful types of cellulose or paper types of substrates. These cellulose or paper types of substrates can be used dry or they can be wetted with water or alcohol or other suitable moistening agent which will assist in the collection of DNA-containing material. Alternatively, the collection area on the subject can be wetted. Other substrates that can be used are sticky or adhesive types of substrates such as Instant Lifter™ manufactured by BVDA, International of Holland or GLUE Dots™ manufactured by Glue Dots International of New Berlin, Wis. or ReTabs® manufactured by Identicator Corporation of Marina Del Rey, Calif. or other adhesive or sticky materials which can be applied to a surface such as the previously mentioned cellulose or paper types of substrates. Other types of sticky substrates which can be used for surface DNA collection are a Micropore™ and Safe-Release™substrates manufactured by 3M of St. Paul, Minn.

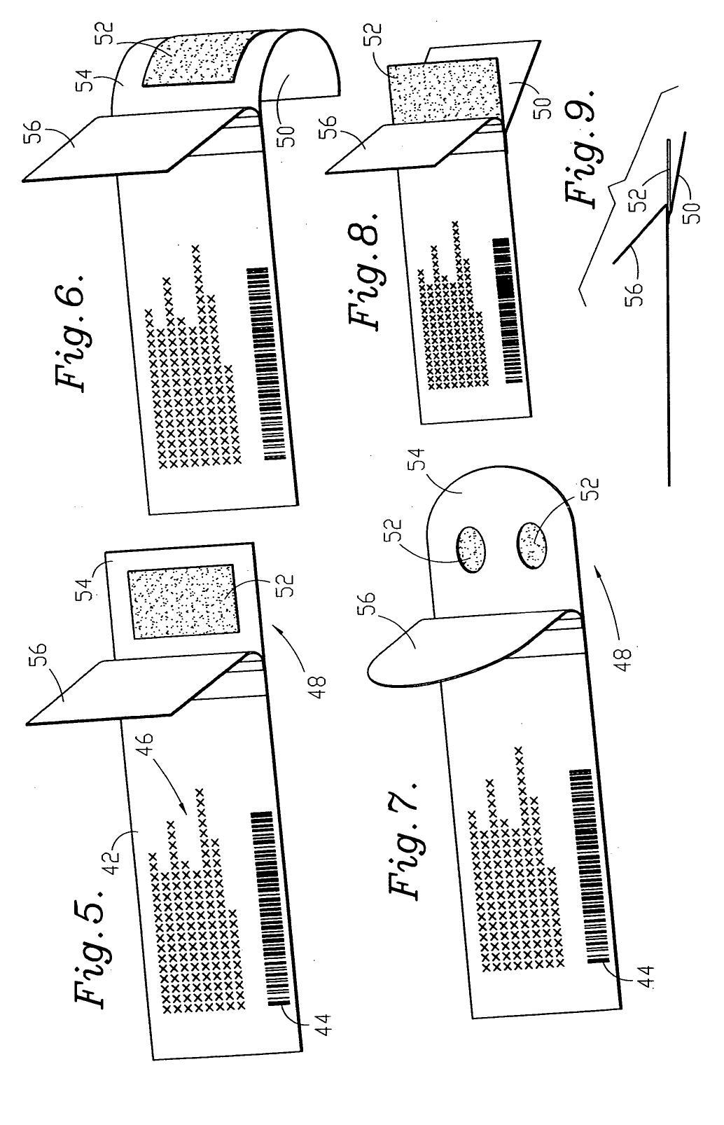

Referring now to FIGS. 5 and 6, an alternative embodiment of a DNA collection device is shown. The embodiment of FIGS. 5 and 6 is particularly suitable for collection of DNA-containing material from the skin surface of a subject. The embodiment of FIGS. 5 and 6 is a swab-type device which can be rubbed on the skin surface for collection of DNA-containing material from the subject's skin.

The device of FIGS. 5 and 6 is of generally similar structure as the device of FIGS. 3 and 4, however, rather than having two collection substrate areas 52 as shown in FIG. 3, the embodiment of FIGS. 5 and 6 is provided with a larger collection substrate area. The device of FIGS. 5 and 6 has generally the same layer configuration as described in FIG. 4, that is, a backing sheet having a collection substrate thereon and a cover sheet having a void therein to expose the collection substrate. As in the embodiment of FIGS. 3 and 4, a data portion 42 is provided having a bar code 44 thereon and an information area 46.

The embodiment of FIGS. 5 and 6 is adapted to present a large surface area collection substrate such as a cellulose or paper-type of collection substrate for wiping on the skin surface to collect DNA-containing material. The collection substrate can be wet or dry as previously described. As is shown in FIG. 6, an effective way of using the embodiment of FIGS. 5 and 6 to collect a DNA sample is to slightly bend the DNA collection portion 48 so that both edges of cover sheet 54 and backing paper 50 which surround collection substrate 52 can be grasped in one hand by the user. The user then rubs the collection substrate 52 against the skin of the subject to gather DNA containing material from the subject.

Referring now to FIG. 7, and alternative embodiment of the present invention is shown which is configured to allow DNA collection in the manner of the embodiment shown in FIGS. 3 and 4 and also to allow comfortable insertion of the DNA collection portion 48 of the embodiment shown in FIG. 7 into the mouth of the subject for saliva collection. In using the embodiment of FIG. 7, the user or operator folds back protective layer 56 and requests that the subject insert DNA collection portion 48 into their mouth. The insertion of the DNA collection portion 48 into the mouth will allow saliva to collect on collection substrate 52, and after a time of collection in the mouth, DNA collection portion 48 can be removed and placed into an envelope for mailing to an appropriate testing facility.

Referring now to FIGS. 8 and 9, yet another alternative embodiment of the present invention is shown. In the embodiment of FIG. 8, cover sheet 54 has been eliminated, and collection substrate 52 is sandwiched between backing 50 and protective layer 56. In the embodiment shown in FIG. 8, collection substrate 52 can be a piece of filter paper or a substance provided with a sticky or adhesive surface. If filter paper is utilized, collection substrate can be rubbed across the skin or used to absorb saliva from the mouth. If an adhesive surface is used, the user or operator can fold back protective layer 56 and rub collection substrate 52 on the skin of the subject. Alternatively, protective layer 56 can be peeled back from collection substrate 52, and the subject could be asked to place their fingertips onto the sticky or adhesive surface of collection substrate 52 to collect DNA-containing material.

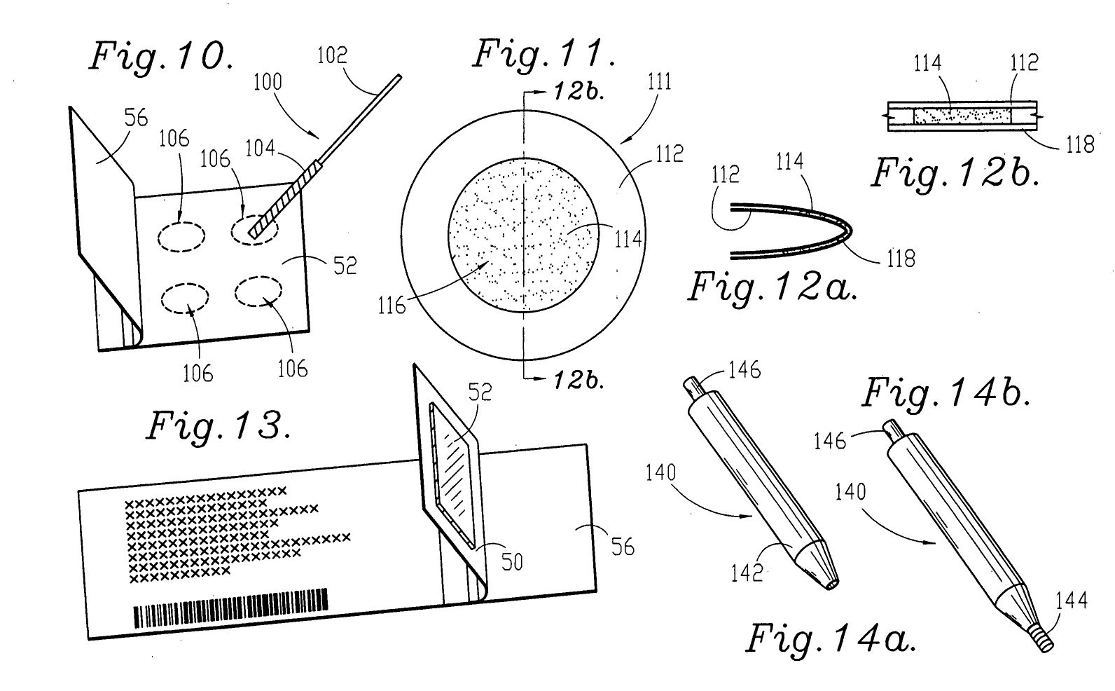

Referring now to FIG. 10, an alternative embodiment of a DNA collection device is shown in which collection substrate 52 is provided with a protective layer 56 and with a second protective layer 56 covering the backside (not shown) of collection substrate 52. To use the embodiment shown in FIG. 10, a collection swab 100 is inserted into a pool of collected saliva or into the mouth of an individual and saliva is collected on absorbent 104 while the swab 100 is held by handle 102. Once absorbent 104 is saturated with saliva, the saliva can be transferred to collection substrate 52 by touching absorbent 104 to collection substrate 52 and allowing the saliva on absorbent 104 to be absorbed by collection substrate 52. For the convenience of the user, application locations 106 are indicated by the use of a printed dotted line on collection substrate 52.

Referring now to FIG. 11, an alternative embodiment of a DNA collection device 111 is shown. Device 111 comprises a cover sheet 112 having a large circular opening or void 116 in the center thereof which allows collection substrate 114 to be accessible therethrough. Collection substrate 114 can be filter paper or another suitable absorbent type of substrate for collecting saliva. Referring now to FIG. 12b, the sandwich-like makeup of device 111 is shown. In FIG. 12b, collection substrate 114 is sandwiched between backing 118 and cover sheet 112. Referring now to FIG. 12a, the manner in which device 111 is used will be described. When it is desired to take a saliva sample of a subject, device 111 is folded in half by the operator to provide a shape as shown in FIG. 12a. The operator will be careful to wear rubber gloves so as to not contaminate device 111 with extraneous DNA. Alternatively, the operator, if absolutely necessary, can be bare-handed but being sure to touch only the outside edge of cover sheet 112 or backing 118 and being careful not to touch the interior portion of backing 118 or collection substrate 114. Once device 111 is folded in half as shown in FIG. 12a, the device is inserted into the mouth of the subject to be tested and allowed to reside in the mouth until saliva has saturated collection substrate 114. After saliva has saturated collection substrate 114, device 111 is withdrawn from the mouth and allowed to dry before shipment. Alternatively, device 111 can be placed in a preservative solution and shipped.

Referring now to FIG. 13, yet another embodiment of a DNA collection device is shown. In the device of FIG. 13, collection substrate 52 is attached to backing 50. Collection substrate 52 could be filter paper or an adhesive-type of collection substance. Collection substrate 52 attached to backing 50 folds down against protective layer 56 when not in use. To utilize the device shown in FIG. 13, backing paper 50 is folded back away from protective layer 56 to expose collection substrate 52. If collection substrate 52 is a filter paper, it may be used wetted or dried. Once collection substrate 52 is exposed, the operator can rub the collection substrate 52 against the skin of a subject to collect DNA containing material from the subject.

Referring now to FIGS. 14a and 14b, yet another embodiment of a DNA collection device is shown. Device 140, shown in FIG. 14a, is a retractable pen-type pen type device which contains within case 142 a collection substrate 144 (FIG. 14b) which can be extended from case 142 by depressing retraction button 146 to extend collection substrate 144 much like a retractable pen or pencil. When collection substrate 144 is extended from case 142, the operator can grasp case 142 and rub collection substrate against the skin of a subject from which a DNA sample is desired. Once the sample has been collected, retraction button 46 can be depressed a second time to retract collection substrate 144 into case 142.

It will be appreciated that the embodiment of FIGS. 14a and 14b can incorporated a number of different embodiments of the collection substrate 144. For example the collection substrate could be an untreated absorbent material or it can be treated to be either sticky or to incorporate a DNA stabilizing agent. Alternatively, substrate 114 can be pre-wetted or be used in a situation in which the collection area of the subject is moistened.

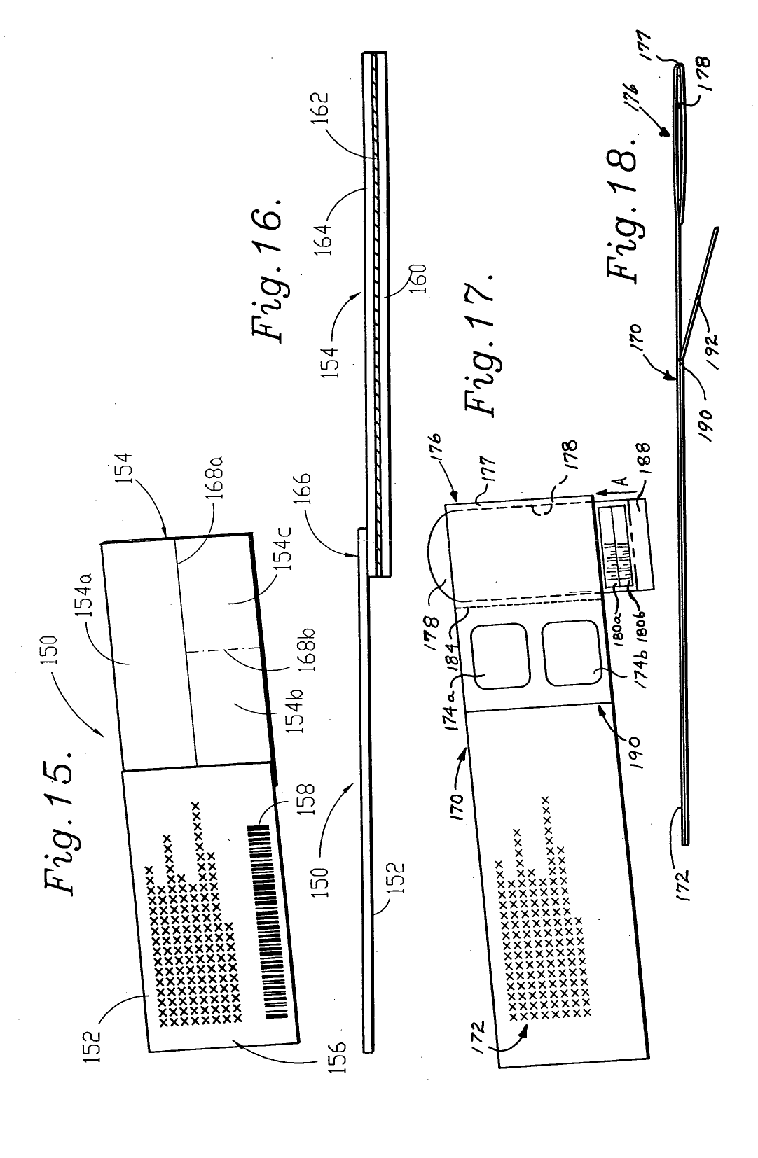

Referring now to FIG. 15, yet another embodiment of a collection device is shown. Device 150 is generally comprised of a data portion 152 and a collection portion 154. As in previous embodiments, data portion 152 is comprised of an information section 156 which can also include a bar code 158. Collection portion 154 is, in this embodiment, a sticky or adhesive-type material which is sandwiched between a backing layer and a protective layer. Referring now to FIG. 16, the structure of device 150 is shown. Collection portion 154 is attached to data portion 152. Collection portion 154 is comprised of a backing material 160 which has applied to it an adhesive or sticky collection substrate 162. Collection substrate 162 is then covered by protective layer 164 to prevent anomalous contamination of collection substrate 162. The three components, the backing material 160, collection substrate 162 and protective layer 164 of collection portion 154 are attached to data portion 152 at junction 166 by glue or tape or other appropriate connecting substance. Referring now to FIG. 15, collection portion 154 can be subdivided into multiple collection portion subsections 154a, 154b, 154c. This subdividing of collection portion 154 allows collection of multiple DNA and fingerprint evidentiary materials from the subject. As shown by dotted lines in FIG. 15, collection portion 154 is subdivided into separable pieces by perforation lines 168 which allow separation of the various subunits 154a, 154b, 154c of collection portion 154 after use of the subunits.

To use device 150 to collect DNA specimens and/or fingerprint specimens of a subject, the user or operator first determines which type of specimen is to be collected, a DNA specimen or a fingerprint specimen. It will be appreciated that with a sufficiently sticky or adhesive substance that the collection of fingerprints from a suspect will also provide a sufficient DNA sample for evaluation. If the operator wishes to collect a DNA sample by rubbing collection substrate 162 across the surface of the skin, the operator peels back protective layer 164 along perforation line 168a to expose collection subportions 154b and 154c. The operator can then rub the skin of a subject with the adhesive collective substrate 162 which is within subportions 154b, 154c. DNA containing material will then adhere to collection substrate 162. Once the DNA sample has been collected on subportions 154b, 154c, the operator can then replace protective layer 164 across subportions 154b, 154c and separate one of sections 154b, 154c along with its associated backing material 160 and protective layer 164 along perforations 168a and 168b. This provides a separated DNA sample portion which may be kept apart from the remainder of device 150. This procedure of maintaining a separate DNA sample with the collecting institution is used in police agencies that wish to maintain an unanalyzed sample as part of the chain of evidence while sending another sample, such as section 154b, to a laboratory for analysis.

If the operator now wishes to collect fingerprints, the operator peels back the upper portion of protective layer 154 which is covering subportion 154a. The operator then applies the subject's finger tips to subportion 154a to leave the fingerprint impressions on collection substrate 162 along with oils from the fingers which could also be utilized for a DNA sample. It will be appreciated by those skilled in the art that a sufficient DNA sample can be obtained from a fingerprint or a thumbprint of an individual when the fingerprint or thumbprint is applied to a sticky or adhesive substance that will trap skin oils and DNA-containing material from the subject.

To use the embodiment of FIG. 15 for collecting a DNA sample from a fingerprint, the user first has the subject apply their fingerprint or thumbprint to a portion of the FIG. 15 embodiment that is intended to receive a DNA sample from the subject. This first print from the subject should be used as the specimen source for DNA analysis. Use of the first print is preferred as it will have a greater amount of skin oil and DNA-containing material than will the subsequent prints from the same thumb or finger. After, the fingerprint or thumbprint that is to be used for the DNA specimen has been obtained, a subsequent fingerprint or thumbprint may then be taken which is to be used for conventional fingerprint records.

Referring now to FIG. 17, yet another embodiment of the present invention is shown. The embodiment of FIG. 17 can be used for fingerprint collection or for saliva collection for a DNA specimen sample, or it can be used to wipe the skin to collect DNA containing material from the surface of the skin. Device 170 is comprised of subject information area 172 where relevant information about the subject from whom the DNA sample is being collected may be recorded. Next to subject information area 172 are fingerprint spaces 174a, 174b. Next to the fingerprint areas is specimen area 176 which is comprised of envelope portion 177 containing collection substrate 178. As with other embodiments of the present invention, collection substrate 178 can be an absorbent material such as one of the previously identified cellulose absorbent papers. Collection substrate 178 can be used in a wet or dry fashion to collect a skin surface sample of DNA containing material or collection substrate 178 can be inserted into the mouth of an individual for collection of DNA containing material from the mouth such as saliva. The use of collection substrate 178 to collect material from the mouth will be described in detail hereinafter. Also located on collection substrate 178 are removable bar code labels 180a, 180b which can be removed from collection substrate 178. One of bar code labels 180a, 180b is placed on device 170 near subject information area 172, and the other bar code label 180a, 180b is placed on specimen area 176, preferably on envelope portion 177. By use of the removable bar code labels, specimen area 176 and the subject information area 172 can be associated with one another after separation of specimen area 176 from subject information area 172.

Still referring to FIG. 17, the use of specimen area 176 will be described. When it is desired to collect a sample using specimen area 176, the user shifts collection substrate 178 upwardly, in the direction of Arrow A, within envelope 177 such that the top portion of collection substrate 178 extends from the top of envelope 177. This presents collection substrate 178 for use in collecting a sample of DNA containing material from the subject. If it is desired to collect a saliva sample, specimen area 176 may be detached from subject information area 172 by tearing along perforated line 184. This is a matter of convenience for both the subject and the operator and allows collection substrate 178 to be placed within the mouth of the subject without having the subject information area 172 dangling from the side of envelope 177 while collection substrate 178 is in the mouth of the subject. Again, once specimen area 176 is separated from subject information area 172, the operator will wish to detach bar code labels 180a, 180b and apply them to subject information area 172 and envelope 177, respectively.

After a saliva sample has been absorbed onto collection substrate 178, the operator will then bend collection substrate foot 188 at a right angle to envelope 177. Once foot 188 is at a right angle to envelope 177, the separated specimen area 176 can be placed on a table top or other surface to allow the saliva on collection substrate 178 to dry. After the saliva has dried, the operator pulls downwardly on foot 188 to retract collection substrate 178 into envelope 177. The user or operator can then place specimen area 176 into a suitable envelope or container for transmitting specimen area 176 to a clinical analysis laboratory for analysis of the DNA in the collected sample.

Alternatively, if specimen area 176 is intended for collecting DNA containing material from the skin surface, collection substrate 178 is extended from envelope 177, and the user, while preferably wearing gloves, applies pressure to one side of collection substrate 178 while wiping the other side of collection substrate 178 over the skin of the subject to collect a DNA-containing specimen. After the specimen is obtained, the user pulls downwardly on foot 188 to retract collection substrate 178 into envelope 177, and specimen area 176 is mailed to a laboratory for analysis.

The embodiment of FIG. 17 also provides for the obtaining fingerprints from the subject. When it is desired to obtain fingerprints from the subject, the thumbs or fingers may be properly inked and applied to areas 174a, 174b. Once the fingerprints are obtained, the fingerprint containing areas 174a, 174b are folded along fold line 190 into the portion of the device upon which subject information area 172 is printed. This portion of the device comprises an envelop, having sealing flap 192 (FIG. 18), which can receive the fingerprint areas 174a, 174b and allow for storage or mailing of the fingerprints to a location of choice. It will be appreciated by those skilled in the art that fingerprint areas 174a, 174b also can be comprised of a sticky or adhesive substance which is capable of recording a fingerprint without the use of ink.

Having now discussed the various embodiments of DNA collection devices it will now be appreciated that any of these devices can be incorporated into a sexual assault evidence kit having a DNA evidence collection device or a DNA and fingerprint evidence collection kit or other DNA evidence collection kit. Now it will be useful to discuss the manner in which extraneous DNA or non-specimen DNA can be eliminated from being an evidentiary issue in the collection of DNA specimen samples where the chain of evidence or custody of evidence must be provable and verifiable and where the possibility of specimen contamination by extraneous DNA or non-specimen DNA must be ruled out.

In the conventional DNA collection kit, the collection kit is comprised of a number of different items. These items include instruction sheets; authorization forms for obtaining samples from the victim; medical history and assault information forms; paper bags for collecting such items as foreign material, the outer clothing of the victim, and the undergarments of the victim; an examination sheet for marking the location of evidence located and samples obtained; a vaginal swab and smears collection kit containing microscope slides in a case and swabs, all in an envelope; an oral swab and smear collection envelope containing a microscope slide in a case and a swab, all in an envelope; a pubic hair combings collection kit containing a paper collection sheet, and a comb, all in an envelope; a debris collection envelope containing a collection paper and scraper device for obtaining dried secretions and fingernail scrapings from the victim, a rectal swab and smear kit containing swabs and a microscope slide in a case for obtaining rectal samples from the victim, all in an envelope; a known saliva specimen sample device in an envelope; a blood samples kit containing vials for obtaining blood samples from the victim with an envelope for holding the vials; and two envelopes for holding hairs pulled from the head and the pubic area. All the above items are assembled into a box or a case or a holder which is then sealed and is not to be opened other than by an authorized person just prior to collecting specimens from a sexual assault victim. One such kit is manufactured by Law Crime Scene Products, North Huntingdon, Pa., and another such kit is manufactured by Lynn Peavey Company, Lenexa, Kans.

Each one of the individual items previously mentioned is touched and handled by a person who is packing the materials into the case or holder during assembly of the kit. The kit packers may or may not have their hands covered by rubber gloves during the packing process, therefore, the potential exists for DNA containing material to be rubbed off the kit packers body and onto the various portions of the DNA evidence collection kit during packing. While individual components of such DNA evidence kits, such as individual swabs, may have been sterilized after being placed into their individual packing with an agent such as ethylene oxide, there has never been any attempt to eliminate the potential for cross-contamination of a collected DNA specimen by the extraneous DNA which may enter the kit during the packing process and which may contaminate an evidence collection kit.

The potential for contamination of a DNA evidence kit is substantial, and the inadvertent inclusion of extraneous DNA from a person other than the subject can lead to inaccurate DNA analysis. The inaccurate DNA analysis is a result of the extraneous DNA being present along with the specimen DNA during the process of replication of the DNA from the specimen to build up the quantity of specimen DNA to a sufficient level to allow analysis. Basically, this procedure is accomplished by multiple replications of the specimen DNA by polymerase chain reaction (PCR) technology or equivalent technology.

It will be appreciated that during this process of replication of specimen DNA that if extraneous DNA is present in the sample, it, too, will be replicated along with the specimen sample, and the presence of the two different DNA samples will provide an incorrect analysis and or unreliable result. The present invention avoids this problem of replication of extraneous DNA by causing any extraneous DNA present in the assembled kit to be rendered incapable of intact strand replication.

To accomplish this treatment of a DNA collection kit to prevent the replication of extraneous DNA which may have entered the kit during packing, the entire packed kit is exposed to an effective quantity of an agent which will disable any DNA present in the DNA specimen kit after it is packed and closed from interfering with subsequent specimen DNA analysis. This disabling of the extraneous DNA is accomplished by exposing the extraneous DNA to an agent which will prevent the double strands of extraneous DNA from separating into intact strands of DNA when the desired specimen DNA is heated during the replication process. Typical agents which can be used to disable DNA in this manner and prevent the extraneous DNA from interfering with subsequent specimen specific DNA analysis are gamma radiation, ethylene oxide, ion beam exposure, X-ray and exposure to electron beam ionization. It will also be appreciated by those skilled in the art that any other means, either physical or chemical, such as other forms of radiation or chemical exposure which will succeed in disabling DNA from interfering with the subsequent DNA specimen analysis can be used within the inventive concept of the present invention.

In the foregoing description, certain terms have been used for brevity, clearness and understanding; but no unnecessary limitations are to be implied therefrom beyond the requirements of the prior art, because such terms are used for descriptive purposes and are intended to be broadly construed. Moreover, the description and illustration of the inventions is by way of example, and the scope of the inventions is not limited to the exact details shown or described.

Certain changes may be made in embodying the above invention, and in the construction thereof, without departing from the spirit and scope of the invention. It is intended that all matter contained in the above description and shown in the accompanying drawings shall be interpreted as illustrative and not meant in a limiting sense.

Having now described the features, discoveries and principles of the invention, the manner in which the inventive devices and method of preventing extraneous DNA from contaminating DNA specimen test results are constructed and used, the characteristics of the construction, and advantageous, new and useful results obtained; the new and useful structures, devices, elements, arrangements, parts and combinations, are set forth in the appended claims.

It is also to be understood that the following claims are intended to cover all of the generic and specific features of the invention herein described, and all statements of the scope of the invention which, as a matter of language, might be said to fall therebetween.

1-95. (canceled)

96. A method of collecting DNA-containing material from a subject comprising providing a sticky collection substrate,
applying the fingerprint surface of the finger or thumb of the subject to said substrate, and
allowing DNA-containing material from the fingerprint surface of the finger or thumb of the subject to adhere to said collection substrate.

97. A method of associating fingerprint evidence obtained from a subject with DNA evidence obtained from a subject comprising:
providing a sticky collection substrate,
applying a fingerprint surface of the finger or thumb of the subject to said substrate to generate a fingerprint of the subject in the substrate,
allowing DNA-containing material from the fingerprint surface of the finger or thumb of the subject to adhere to said collection substrate, and
recording an image of the generated fingerprint of the subject prior to use of the collection substrate for DNA analysis.

98. A method of collecting fingerprint evidence from a subject and DNA evidence from a subject comprising:
providing plurality of a sticky collection substrate portions,
applying a fingerprint surface of a finger or thumb of the subject to a first collection substrate portion to generate a first fingerprint in said first substrate portion,
reapplying said fingerprint surface of said finger or thumb to a second substrate portion to generate a second fingerprint in said second substrate portion, and
using said one of said first or second fingerprints in said first or second substrate portions for analysis of DNA-containing material collected from said fingerprint surface.

99. A method of avoiding prejudice of forensic specimen testing results at a forensic testing laboratory comprising:
providing a specimen collection device, said device having a specimen collection portion and a subject information portion,
obtaining a forensic specimen from a subject with said specimen collection portion,
applying a first barcoded label to said specimen collection portion,
applying a second barcoded label to said subject information portion, said second barcoded label having an identical barcode to said first barcoded label,
transmitting said specimen collection portion with said first barcode to a laboratory for testing
retaining said subject information portion.

100. The method as claimed in claim 99 wherein said forensic specimen is free DNA or DNA-containing material.

101. The method as claimed in claim 99 wherein said forensic specimen is a fingerprint or thumbprint.

102. The method as claimed in claim 99 wherein said forensic specimen is urine or other body fluid.
